(19) United States
(12) Patent Application Publication
(10) Pub. No.: US 2026/0174421 A1
(43) Pub. Date: Jun. 25, 2026

(54) STERNAL STRUTS

(71) Applicant: Mayo Foundation for Medical Education and Research, Rochester, MN (US)

(72) Inventors: Nathaniel L. Robinson, Rochester, MN (US); Juan A. Crestanello, Rochester, MN (US); Jonathan M. Morris, Rochester, MN (US)

(21) Appl. No.: 19/427,184

(22) Filed: Dec. 19, 2025

Related U.S. Application Data
(60) Provisional application No. 63/737,228, filed on Dec. 20, 2024.

(51) Int. Cl. A61B 17/02; A61B 17/00

(52) U.S. Cl. CPC A61B 17/02; A61B 2017/00526

(57) ABSTRACT

A strut that can be used to prop open a patient's chest wall in connection with delayed sternal closure procedures. Embodiments comprise a one-piece body including a bridge portion having first and second opposite end portions, a first engaging portion on the first end portion of the bridge portion, and a second engaging portion on the second end portion of the bridge portion. The first engaging portion is configured to engage a first side of a patient's chest wall. The second engaging portion configured to engage a second side of the patient's chest wall. Embodiments also comprise a kit including a plurality of the struts, wherein two or more of the plurality of struts have bridge portions defining different lengths.

### REFERENCE TO RELATED APPLICATION

This application claims the benefit of U.S. Provisional Application No. 63/737,228, filed on Dec. 20, 2024, and entitled Sternal Struts, which is incorporated herein by reference in its entirety and for all purposes.

### TECHNICAL FIELD

This disclosure relates generally to sternal retractors and related methods.

### BACKGROUND

Sternal or thoracic retractors are used during surgical procedures to provide an opening in a patient's chest wall, typically by separating and opening the patient's sternum and/or ribs, to provide access into the chest cavity. Retractors of these types are disclosed, for example, in the following patent publications: U.S. Pat. No. 7,507,202; U.S. Pat. App. Pub. 2015/0327847; CN pub. 204147069; CN pub. 105997166; CN pub. 112971880. Following surgical procedures in the chest cavity, sternal closure devices such as those commercially available from Peters Surgical or Pusula Medikal may be used to assist with the reapproximation and closure of the cavity.

There are instances when the sternum and ribs may not be immediately reapproximated and closed following these procedures. Examples of such instances include those where there is ongoing bleeding, edema, infection, or hemodynamic instability from compression of the heart by the chest wall. Delayed sternal closure procedures may be performed to maintain the chest wall open in instances such as these. Devices used to keep the chest wall open during delayed sternal closure procedures include plastic devices hand formed in the operating room (OR), including syringes with notches cut into them by surgeons, and sternal plates that can be screwed to the bones. Patients may be transported from the OR to an intensive care unit (ICU) for medical resuscitation while their sternums are kept open by devices of these types, after which they are taken back to the OR for delayed sternal closure one or several days later.

There remains a continuing need for improved devices and methods for use in connection with delayed sternal closure procedures. Such devices and methods that can effectively maintain the chest cavity in an open state, while accommodating a range of patient sizes and dimensions, would be advantageous. Such devices and methods that can be relatively easily placed and removed, and with minimal trauma to the patient, would be especially desirable.

### SUMMARY

Disclosed embodiments include struts for use in connection with delayed sternal closure procedures, and related methods. The struts can be customized or otherwise configured to accommodate ranges of patient sizes and dimensions. The struts can be relatively easily placed and removed, with minimal patient trauma. They effectively and securely maintain the chest wall in an open state when in place.

A first example is a strut, such as a sternal strut, comprising: a one-piece body, including: a bridge portion having first and second opposite end portions; a first engaging portion on the first end portion of the bridge portion, the first engaging portion configure to engage a first side of a patient's chest wall; and a second engaging portion on the second end portion of the bridge portion, the second engaging portion configured to engage a second side of the patient's chest wall.

In some embodiments, each of the first and second engaging portions includes a concave portion. The bridge portion may include a first side, and wherein the first side of the bridge portion is generally planar. The first engaging portion may include a first lip. The first lip of the first engaging portion may be generally planar. The first lip of the first engaging portion may be coplanar with the first side of the bridge portion. The second engaging portion may include a first lip, and wherein the first lip of the second engaging portion may be generally planar and coplanar with the first side of the bridge portion. The first engaging portion may include a second lip, and wherein the first and second lips of the first engaging portion may be spaced apart by a first distance and define a chest wall-engaging recess; and the second engaging portion may include second lip, and where the first and second lips of the second engaging portion may be spaced apart by a second distance and define a chest wall-engaging recess; and the bridge portion may include a second side, and wherein at least portions of the second side of the bridge portion may be spaced apart from the first side of the bridge portion by a third distance that is less than the first and second distances. The first and second distances may be about equal to one another. The second side of the bridge portion may include a concave portion. The first and second lips of the first engaging portion may be parallel to one another; and the first and second lips of the second engaging portion may be parallel to one another.

In some embodiments, the first engaging portion includes a first lip and second lip, and wherein the first and second lips of the first engaging portion may be spaced apart by a first distance and define a chest wall-engaging recess; and the second engaging portion includes a first lip and a second lip, and where the first and second lips of the second engaging portion may be spaced apart by a second distance and define a chest wall-engaging recess. For example the bridge portion may include first and second opposite sides, and at least portions of the second side of the bridge portion may be spaced apart from the first side of the bridge portion by a third distance that is less than the first and second distances. The first and second lips of the first engaging portion may be parallel to one another; and the first and second lips of the second engaging portion may be parallel to one another.

In some embodiments, the strut comprises one or more suture apertures, wherein the one or more suture apertures are through one or more of the bridge portion, the first engaging portion or the second engaging portion.

In any or all of the above embodiments of the first example, the bridge portion defines a length between the first and second engaging portions.

A second example is a kit including a plurality of struts in accordance with any or all embodiments of the first example, wherein two or more the plurality of struts have bridge portions defining different lengths. In some embodiments the kit of the second example includes packaging containing the plurality of struts.

A third example is a method for using the kit of any or all embodiments of the second example, including selecting one of the two or more of the plurality of struts. Some embodiments of the third example may include inserting the selected strut into the patient's chest wall.

A fourth example is a method for propping open the patient's chest wall using a strut in accordance with any or all embodiments of the first example, comprising inserting the strut into the patient's chest wall with the second side of the bridge portion facing the patient.

A fifth example is a strut, such as a sternal strut, comprising: a body, including: a bridge portion having first and second opposite end portions and first and second opposite sides; a first engaging portion on the first end portion of the bridge portion, the first engaging portion defining a concave opening having a first distance dimension and configured to engage a first side of a patient's chest wall; and a second engaging portion on the second end portion of the bridge portion, the second engaging portion defining a concave opening having a second distance dimension and configured to engage a second side of the patient's chest wall; and wherein at least portions of the second side of the bridge portion are spaced apart from the first side of the bridge portion by a third distance dimension that is less than the first and second distance dimensions.

In some embodiments, the first and second distance dimensions are about equal to one another.

In some embodiments, the second side of the bridge portion includes a concave portion.

In any or all of the above embodiments of the fifth example, the body comprises a one-piece member.

A sixth example is a strut kit, such as a sternal strut kit, comprising: a plurality of bridge members, each having first and second opposite end portions, and wherein the plurality of bridge members have different lengths; a first engaging member including first and second opposite end portions, wherein: the first end portion is configured to engage a first side of a patient's chest wall; and the second end portion includes a bridge member-receiving structure configured to receive the first end portion of any one of the plurality of bridge members; and a second engaging member including first and second opposite end portions, wherein: the first end portion is configured to engage a second side of the patient's chest wall; and the second end portion includes a bridge member-receiving structure configured to receive the second end portion of any one of the plurality of bridge members, and to allow a distance between the first end portion of the first engaging member and the first end portion of the second engaging member to be adjusted.

In some embodiments, at least the first and second opposite end portions of each of the plurality of bridge members comprises a threaded shaft; and the bridge member-receiving structures of the second end portions of each of the first and second engaging members comprises a threaded bore.

In some embodiments, the first end portions of each of the first and second engaging members includes a concave portion.

Any or all embodiments of the sixth example may also include packaging containing the plurality of bridge members, the first engaging member, and the second engaging member.

A seventh example is a method for assembling a strut from a strut kit in accordance with any or all embodiments of the sixth example, comprising: selecting one of the plurality of bridge members; attaching the first end portion of the selected bridge member to the bridge member-receiving structure of the first engaging member; attaching the second end portion of the selected bridge member to the bridge member-receiving structure of the second engaging member; and adjusting a distance between the first engaging member and the second engaging member by manipulating the location of the bridge member with respect to the bridge member-receiving structure of the second engaging member.

### BRIEF DESCRIPTION OF THE DRAWINGS

FIG. 1 is an isometric illustration of a sternal strut, in accordance with embodiments.

FIGS. 2A, 2B and 2C are top, side and end views, respectively of the sternal strut shown in FIG. 1.

FIG. 3 is diagrammatic illustration of a kit including a plurality of sternal struts in a package, accordance with embodiments, where at least some of the struts have different sizes.

FIG. 4 is a diagrammatic illustration of a sternal strut such as that shown in FIG. 1 placed between edges of a cut and opened sternum of a patient.

FIG. 5 is a diagrammatic isometric illustration of a sternal strut, shown in a first length configuration, in accordance with embodiments.

FIG. 6 is a diagrammatic sectional side view of the sternal strut shown in FIG. 5.

FIG. 7 is a diagrammatic isometric illustration of a sternal strut, shown in a second length configuration, in accordance with embodiments.

FIG. 8 is a diagrammatic sectional side view of the sternal struct shown in FIG. 7.

FIG. 9 is an exploded isometric illustration of the sternal strut shown in FIGS. 5-8.

FIG. 10 is a diagrammatic illustration of a kit including components of the sternal strut shown in FIGS. 5-8, including a plurality of bridge members, in a package, in accordance with embodiments.

FIG. 11 is a diagrammatic illustration of a sternal strut such as that shown in FIG. 5 placed between edges of a cut and opened sternum of a patient.

While the disclosure is amenable to various modifications and alternative forms, specific embodiments have been shown by way of example in the drawings and are described in detail below. The disclosure, however, is not limited to the particular embodiments described. On the contrary, the disclosure is intended to cover all modifications, equivalents, and alternatives falling within the scope of the disclosure as defined by the appended claims.

### DETAILED DESCRIPTION

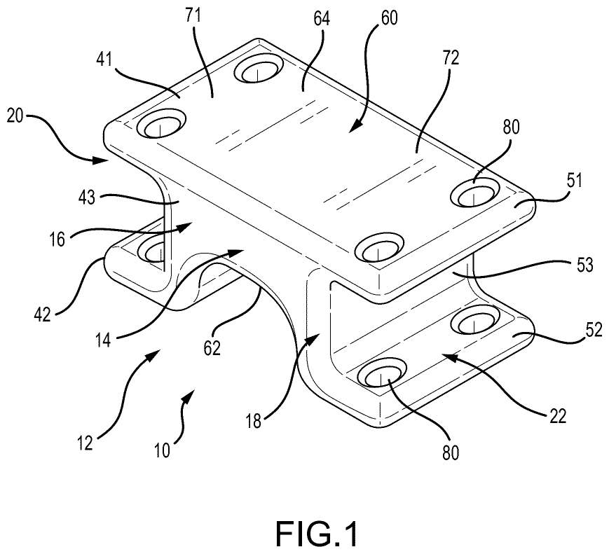

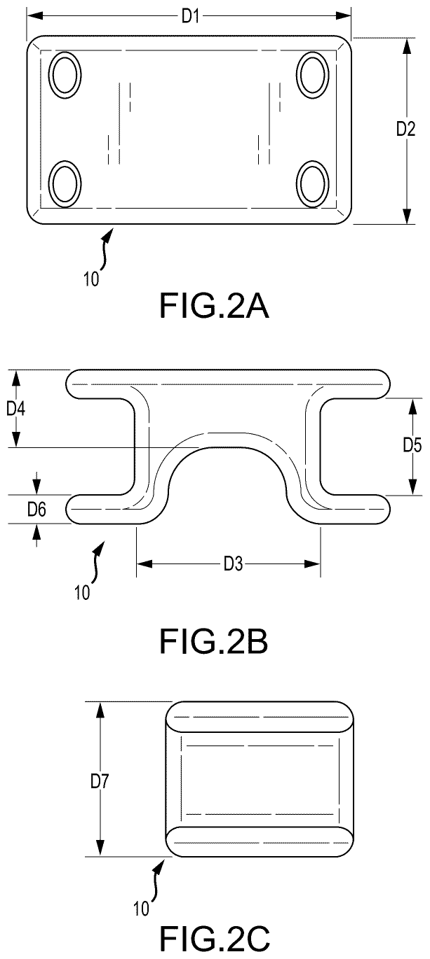

FIG. 1 is an isometric view of a sternal strut 10 in accordance with embodiments. FIGS. 2A, 2B and 2C are top or anterior-facing, side, and end views, respectively, of embodiments of the strut 10. As shown, the strut 10 comprises a body 12 that includes a bridge portion 14 having a first end portion 16 and a second end portion 18. A first engaging portion 20 is located on the first end portion 16 of the bridge portion 14, and a second engaging portion 22 is located on the second end portion 18 of the bridge portion. As described in greater detail below, the strut 10 is configured to maintain portions of the patient's chest wall open for surgical assess when positioned in an opening in the chest wall. In connection with this functionality, the first and second engaging portions 20 and 22 are configured to engage an opening defined by portions of the patient's chest wall when positioned in the opening, such as for example one or more of the patient's sternum or one or more ribs, and/or one or more sides of the patient's sternum if the sternum is split and opened to provide the opening in the chest wall. Because of the bridge portion 14 between the first and second engaging portions 20 and 22, the strut 10 can maintain or prop open the chest wall, thereby providing access into the chest.

As another use case application, the strut 10 can be used to facilitate closure of the patient's chest wall. For example, there are instances when a patient's chest wall has been left open for a period of time and begins to stay open on its own. When closing an opening during instances of these types, a surgeon may use a strut, perhaps a shorter length strut, attach a first end of the strut to a first side of the chest wall (e.g., by sutures), push the chest wall toward its closed position and the second end of the strut, and attach the second end of the strut to the second side of the chest wall. In this application the strut 10 may help keep the sides of the chest wall closer together for a period of time in preparation for a full closure.

The illustrated embodiments of the first engaging portion 20 include first and second spaced-apart lips 41 and 42, respectively, extending from a base portion 43 on the first end portion 16 of the bridge portion 14. The lips 41, 42 and the base portion 43 define a generally concave channel oriented and sized to receive the first side of the sternum and/or ribs and/or other portions of the patient's opened chest wall when the strut 10 is placed. Similarly, the second engaging portion 22 includes first and second spaced-apart lips 51 and 52, respectively, extending from a base portion 53 on the second end portion 18 of the bridge portion 14. The lips 51, 52 and the base portion 53 define a generally concave channel oriented and sized to receive the second side of the sternum and/or ribs and/or other portions of the patient's opened chest wall when the strut 10 is placed in the patient. The channels of the first and second engaging portions 20, 22 are generally elongated in the illustrated embodiments, and have a longitudinal axis or dimension that, as described below, will be generally parallel with a proximal-distal axis of the opened chest wall when the strut 10 is interested into the patient.

The strut 10 includes a first or top side 60 and a second or bottom side 62. In the illustrated embodiments the top side 60 is configured to be located in an anterior-facing orientation or direction with respect to the patient when the strut 10 is in placed in the patient. The first or top side 64 of the bridge portion 14 is generally planar in the illustrated embodiments. The first or top sides 71 and 72 of the first lips 41 and 51, respectively, are also generally planar in the illustrated embodiments, and in these particular embodiments are also shown as being coplanar with the top side 64 of the bridge portion 14. The second lips 42 and 52 are parallel to first lips 41 and 51, respectively, in the illustrated embodiments, and may have planar bottom sides. The interior surfaces of the channels defined by the structures of the first and second engaging portions 20,22, can be relatively smooth to prevent or minimize trauma to the patient's chest wall.

Although the first and second engaging portions 20, 22 have the same size and shape in the illustrated embodiments, other embodiments of the strut 10 may include first and second engaging portions that are configured differently. For example, the first and second lips 41 and 42 of the first engaging portion 20 need not be parallel to one another, and may for example diverge from one another with increasing distance from the base portion 43. The first and second lips 51 and 52 of the second engaging portion 22 need not be parallel to one another, and may for example diverge from one another with increasing distance from the base portion 53. As another example, in other embodiments the first and second engaging portions 20 and 22 have different sizes, shapes and/or orientations.

In the illustrated embodiments, the bottom side 62 is configured to be located in a posterior-facing orientation or direction with respect to the patient when the strut 10 is in placed in the patient. The second or bottom side 62 of the illustrated embodiments is generally concave. Distances between the top side 60 and bottom side 62 (e.g., thicknesses) of the bridge portion 14 at its central portion are therefore less than the distances between the top and bottom sides at the first and second end portions 16 and 18, respectively.

The illustrated embodiments of the strut 10 also includes a plurality of suture apertures 80 on each of the first and second lips 41 and 42 of the first engaging portion 20, and on each of the first and second lips 51 and 52 of the second engaging portion 22. Suture apertures 80 are structures that can be used couple sutures or other attachment structures to the strut 10, and to secure the strut 10 to the patient. Other embodiments of the strut 10 may include more or fewer (for example no) attachment structures such as suture apertures 80, or such attachment structures at other locations such as the bridge portion 14.

FIGS. 2A-2C illustrate dimensions of certain features of an exemplary embodiment of strut 10. This particular embodiment may have an overall length D1 of about 70 mm, and a width D2 of about 40 mm. The length D3 of the bridge portion 14 (e.g., the distance that the chest wall will be opened when the strut 10 is placed in the patient) is about 40 mm. The thickness D4 of the bridged portion 14 at its thinnest location is about 16.5 mm. The distances D5 between the lips 41, 42 of the first engaging portion 20 and the lips 51, 52 of the second engaging portion 22 are about 21 mm. The lips 41, 42, 51 and 52 have thicknesses D6 of about 6 mm. The overall thickness or height D7 is about 33 mm.

Other embodiments of struts such as 10 have other distances for one or more, or all, of the dimensions D1-D7. For example, other embodiments of struts such as 10 that are configured to maintain larger chest openings (e.g., for larger patients), can have larger lengths D3 of the bridge portions, such as for example about 70 mm or about 100 mm.

In the embodiments shown in FIGS. 1 and 2A-2C, the body 12 is manufactured as a 1-piece member. Injection or other molding, 3D printing, and machining from a block of stock material, are examples of manufacturing approaches that can be used to make the struts 10. In other embodiments the struts 10 can be assembled from two or more component parts (e.g., by adhesives or fasteners) that can be separately manufactured by approaches such as those described above. Edges or corners of the struts 10, such as the edges of the outer perimeters of the lips 41, 42, 51, 52 and base portions 43, 53 can be round, chamfered or otherwise provided with minimized or no sharp edges to reduce trauma to the bones or other portions of the patient. Chamfered edges or corners on portions of the struts 10 configured to face the patient when positioned in the opened chest wall may be especially advantageous. Suitable materials for the struts include biocompatible polymers and metals. The struts 10 can be configured for one-time and disposable use, or for sterilization and reuse.

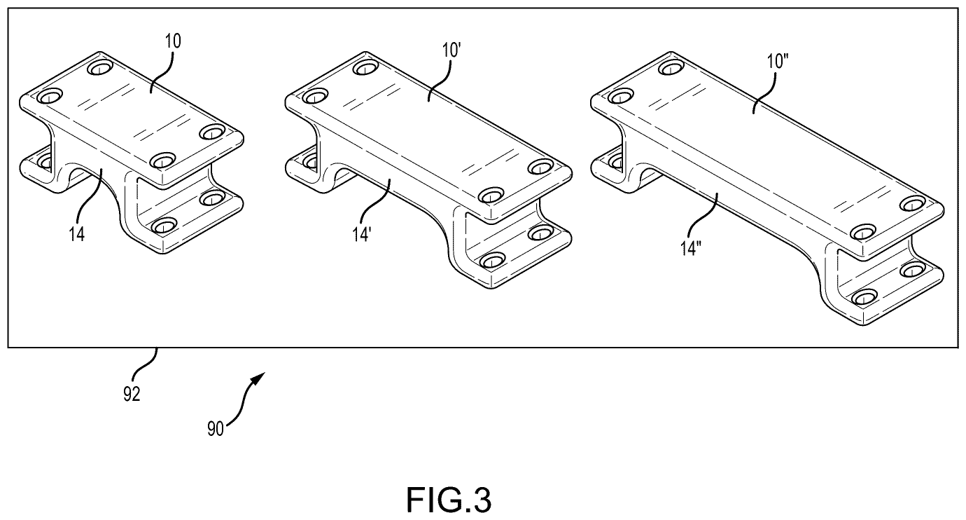

FIG. 3 illustrates a kit 90 including a package 92 containing a plurality of struts 10, 10′ and 10″. Two or more, or all, of the struts 10, 10′ and 10″ are differently sized. For example, in the illustrated embodiments of kit 90 the struts 10, 10′ and 10″ all have bridge portions 14, 14′ and 14″, respectively of different lengths (e.g., 40 mm, 70 mm and 100mm). Other embodiments of kits such as 90 have different numbers of struts such as 10, and/or struts having different dimensions.

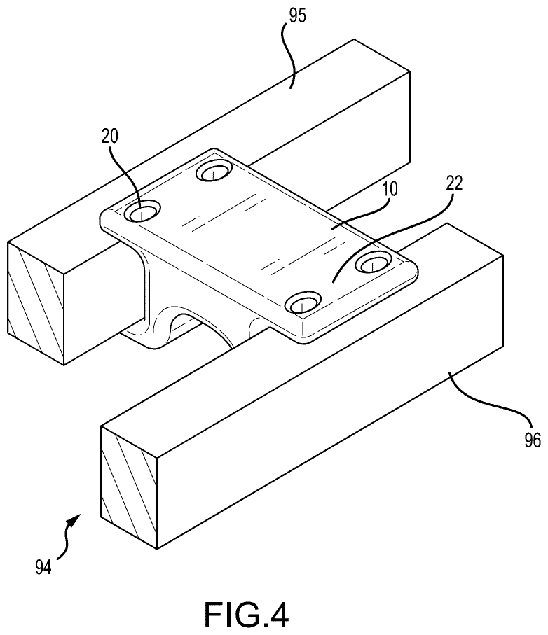

FIG. 4 is a diagrammatic illustration of a strut 10 placed in a patient 94 to maintain an opening in the patient's chest wall. As shown in this example, the patient's sternum is cut and split into right and left sections 95 and 96, and opened, or spread apart. The strut 10 is placed between the right and left sections 95 and 96 of the sternum, with the first engaging portion 20 of the strut engaged with the right section, and the second engaging portion 22 of the strut engaged with the left section. In connection with a delayed sternal closure procedure, a surgeon or operating room staff can access and open a package such as 92 described above containing a plurality of differently sized struts, and select a strut having a size suitable for the procedure (e.g., based on the size of the patient and/or the size of the opening. The selected strut can then be placed into the patient as shown for example in FIG. 4.

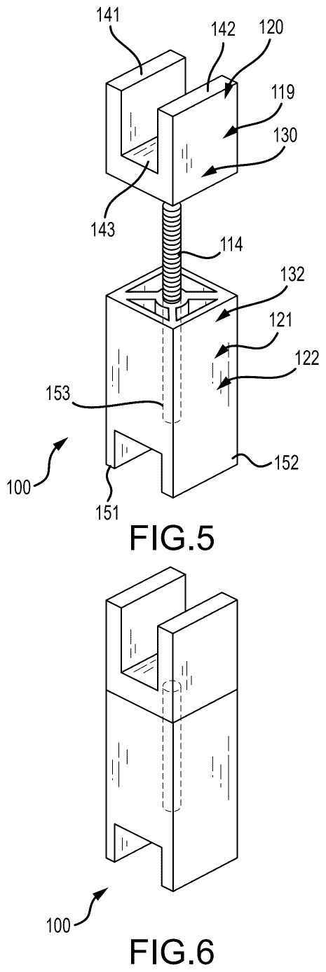

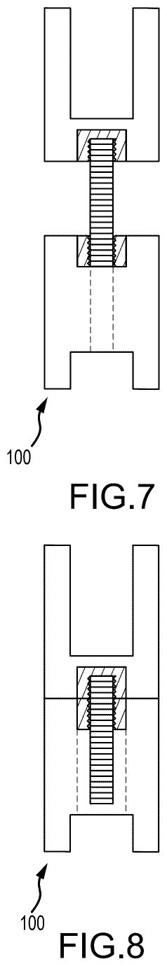

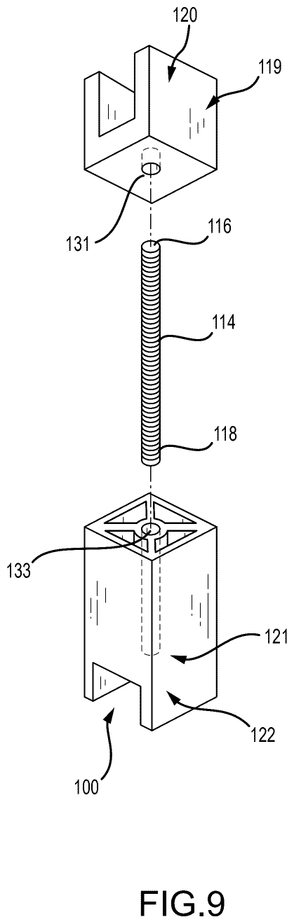

FIGS. 5 and 7 are illustrations of a sternal strut 100 in accordance with embodiments. FIG. 5 shows the strut 100 in a first length configuration, and FIG. 7 shows the strut in a second length configuration, having a length that is shorter that the length of the strut in the first length configuration. FIGS. 6 and 8 are sectional views of the strut 100 in the first and second length configurations, respectively. FIG. 9 is an exploded illustration of the strut 100. As shown, the strut 100 includes a bridge member 114, a first engaging member 119 and a second engaging member 121. Bridge member 114 defines a length, and includes a first end portion 116 and a second, opposite end portion 118. The first engaging member 119 includes a first end portion configured as a first engaging portion 120 to engage a first side of a patient's sternum or other portions of the chest wall, and a second, opposite end portion configured as a bridge member-receiving structure 130 to receive the first end portion 116 of the bridge member 114. The second engaging member 121 includes a first end portion configured as a second engaging portion 122 to engage a second side of a patient's sternum or other portions of the chest wall, and a second, opposite end portion configured as a bridge member-receiving structure 132 to receive the second end portion 118 of the bridge member 114.

The illustrated embodiments of the first engaging portion 120 include first and second spaced-apart lips 141 and 142, respectively, extending from the base portion 143. The lips 141 and 142, and the base portion 143, define a generally concave channel sized to receive the first side of the sternum and/or ribs and/or other portions of the patient's opened chest wall with the strut 100 is placed. Similarly, the second engaging portion 122 includes first and second spaced-apart lips 151 and 152, respectively, extending from a base portion 153. The lips 151 and 152, and the base portion 153, define a generally concave channel sized to receive the second side of the sternum and/or ribs and/or other portions of the patient's opened chest wall when the strut 100 is placed in the patient. The channels of the first and second engaging portions 120, 122 are generally elongated in the illustrated embodiments, and have a longitudinal axis or dimension that, as described below, will be generally parallel with a proximal-distal axis of the opened chest wall when the strut 100 is interested into the patient.

In the illustrated embodiments, the bridge member 114 includes a threaded shaft or rod, and its first end portion 116 and second end portion 118 include threads. Consistent with this embodiment of the bridge member 114, the bridge member-receiving structure 130 of the first engaging member 119 includes a complementary threaded bore 131 configured to receive the threaded first end portion 116 of the bridge member 114, and the bridge member-receiving structure 132 of the second engaging member 121 includes a threaded bore 133. The first end portion 116 of the bridge member 114 can be attached to the first engaging member 119 by screwing the first end portion into the threaded bore 131. When attached in this manner, the first engaging member 119 is fixedly attached to the bridge member 114 at a location along the length of the bridge member 114 defined by the threaded coupling. The second end portion 118 of the bridge member 114 can be attached to the second engaging member 121 by screwing the second end portion into the complementary threaded bore 133. When attached in this manner, the second engaging member 121 is fixedly attached to the bridge member 114 at a location along the length of the bridge member defined by the threaded coupling.

In the illustrated embodiments, the bridge member-receiving structure 132 and the bridge member 114 are configured to allow spacing between the first engaging portion 120 of the first engaging member 119 and the second engaging portion 122 of the second engaging member 121 to be adjustable. The strut 100 can thereby be adjusted to set the distance or spacing length between the first engaging portion 120 and the second engaging portion 122 at a distance or length that it is desired to separate the first and second portions of the patient's chest wall. In the illustrated embodiments of the strut 100, the distance between the first engaging portion 120 and the second engaging portion 122 can be adjustably set or determined by the length of the bridge member 114, and/or the depth to which the bridge member, and for example its second end portion 118, is screwed into the threaded bore 133 of the bridge member-receiving structure 132.

FIGS. 7 and 8, for example, show the strut 100 in a second configuration with the second end portion 118 of the bridge member 114 screwed relatively far into the threaded bore 133 to locate the first engaging member 119 and the second engaging member 121 close to or in contact with one another. In the configuration shown in FIGS. 7 and 8, the distance between the first engaging portion 120 and second engaging portion 122 of the strut is relatively short (e.g., a second spacing length). In contrast, FIGS. 5 and 6 show the strut 100 in a first configuration with the second end portion 118 of the bridge member 144 screwed into the threaded bore 133 by an amount less than that shown in FIGS. 7 and 8, to locate the first engaging member 119 and the second engaging member 121 at a greater spaced-apart distance with respect to one another than the in the second configuration. In the configuration shown in FIGS. 5 and 6, the distance between the first engaging portion 120 and the second engaging portion 122 of the strut is relatively long with respect to that of the second configuration (e.g., a first spacing length greater than the second spacing length).

In the illustrated embodiments, the range of adjustability and spacing lengths that can be provided by the strut 100 are determined by factors such as the depth or length of the threaded bore 133, the length of the bridge member-receiving structure 132 of the second engaging member 121, and/or the length of the bridge member 114. For example, bridge members 114 of different lengths can be configured to provide a range of spacing lengths.

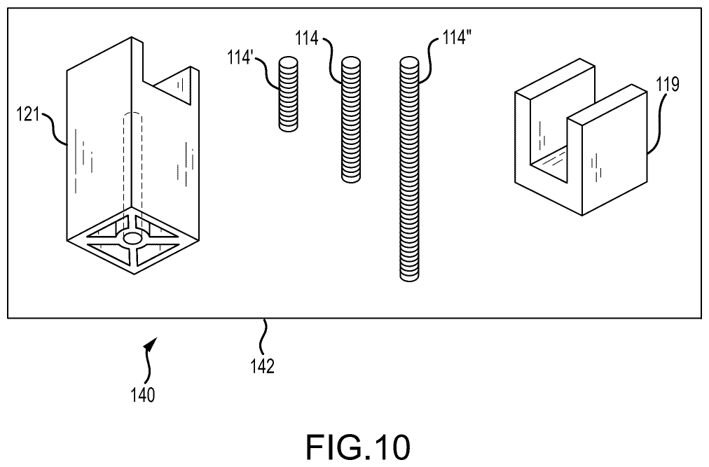

FIG. 10 illustrates a kit 140 including a package 142 containing a plurality of strut components such as a plurality (two are shown for purposes of example) of engaging members such as a first engaging member 119 and a second engaging member 121 of the types described above, and a plurality (three are shown for purposes of example) of bridge members, such as a bridge member 114 of the type described above, and bridge members 114′ and 114″. As shown, at least two of the bridge members 114, 114′ and 114″ have different lengths (all three bridge members 114, 114′ and 114″ have different lengths in the embodiments of kit 140 shown in FIG. 10.

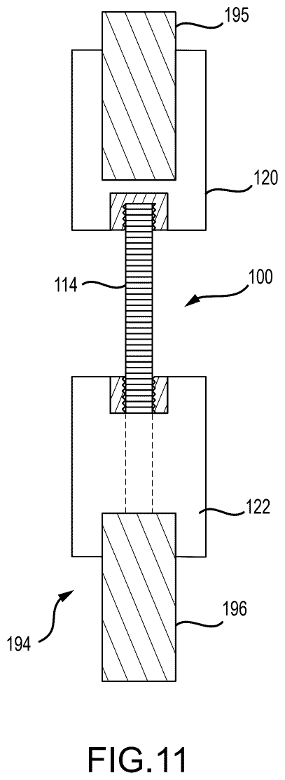

FIG. 11 is a diagrammatic illustration of a strut 100 placed in a patient 194. As shown, the patient's sternum is cut and split into right and left sections 195 and 196, and opened or spread apart. The strut 100 is placed between the right and left sections 195 and 196 of the sternum, with the first engaging portion 120 of the strut engaged with the right section, and the second engaging portion 122 of the strut engaged with the left section. In connection with a delayed sternal closure procedure, a surgeon or operating room staff can access and open a package such as 194 described above, select a pair of engaging members such as the first engaging member 119 and the second engaging member 121, and a bridge member such as bridge member 114, suitable for the procedure (e.g., based on the size of the patient and/or the size of the opening). The selected components can then be assembled to form a strut such as 100 and adjusted to the desired spacing length. The assembled and adjusted strut 100 can then be placed into the patient as shown for example in FIG. 11.

Sternal struts such as 10 and 100 offer important advantages. They are convenient to use, solve a need in the market not yet addressed, and effectively and securely maintain the patient's chest wall in an open state, with the sternum stabilized, as may be needed. They provide little or no trauma to the patient. They provide a safe, customizable, and efficient methodology to maintain an opening in the chest wall when a delayed sternal closure is clinically indicated.

It is to be understood that the above description is intended to be illustrative, and not restrictive. Many other embodiments will be apparent to those of skill in the art upon reading and understanding the above description. It is contemplated that features described in association with one embodiment are optionally employed in addition or as an alternative to features described in or associated with another embodiment. The scope of the invention should, therefore, be determined with reference to the appended claims, along with the full scope of equivalents to which such claims are entitled.

1. A strut, comprising:
a one-piece body, including:
a bridge portion having first and second opposite end portions;
a first engaging portion on the first end portion of the bridge portion, the first engaging portion configured to engage a first side of a patient's chest wall; and
a second engaging portion on the second end portion of the bridge portion, the second engaging portion configured to engage a second side of the patient's chest wall.

2. The strut of claim 1, wherein each of the first and second engaging portions includes a concave portion.

3. The strut of claim 2, wherein the bridge portion includes a first side, and wherein the first side of the bridge portion is generally planar.

4. The strut of claim 3, wherein the first engaging portion includes a first lip.

5. The strut of claim 4, wherein the first lip of the first engaging portion is generally planar.

6. The strut of claim 5, wherein the first lip of the first engaging portion is coplanar with the first side of the bridge portion.

7. The strut of claim 6, wherein the second engaging portion includes a first lip, and wherein the first lip of the second engaging portion is generally planar and coplanar with the first side of the bridge portion.

8. The strut of claim 7, wherein:
the first engaging portion includes a second lip, and wherein the first and second lips of the first engaging portion are spaced apart by a first distance and define a chest wall-engaging recess; and
the second engaging portion includes second lip, and where the first and second lips of the second engaging portion are spaced apart by a second distance and define a chest wall-engaging recess; and
the bridge portion includes a second side, and wherein at least portions of the second side of the bridge portion are spaced apart from the first side of the bridge portion by a third distance that is less than the first and second distances.

9. The strut of claim 8, wherein the first and second distances are about equal to one another.

10. The strut of claim 9, wherein the second side of the bridge portion includes a concave portion.

11. The strut of claim 8, wherein:
the first and second lips of the first engaging portion are parallel to one another; and
the first and second lips of the second engaging portion are parallel to one another.

12. The strut of claim 1, wherein:
the first engaging portion includes a first lip and second lip, and wherein the first and second lips of the first engaging portion are spaced apart by a first distance and define a chest wall-engaging recess; and
the second engaging portion includes a first lip and a second lip, and where the first and second lips of the second engaging portion are spaced apart by a second distance and define a chest wall-engaging recess.

13. The strut of claim 12, wherein the bridge portion includes first and second opposite sides, and at least portions of the second side of the bridge portion are spaced apart from the first side of the bridge portion by a third distance that is less than the first and second distances.

14. The strut of claim 1, wherein the bridge portion defines a length between the first and second engaging portions.

15. A kit including a plurality of struts in accordance with claim 16, wherein two or more of the plurality of struts have bridge portions defining different lengths.

16. A strut kit, comprising:
a plurality of bridge members, each having first and second opposite end portions, and wherein the plurality of bridge members have different lengths;
a first engaging member including first and second opposite end portions, wherein:
the first end portion is configured to engage a first side of a patient's chest wall; and
the second end portion includes a bridge member-receiving structure configured to receive the first end portion of any one of the plurality of bridge members; and
a second engaging member including first and second opposite end portions, wherein:
the first end portion is configured to engage a second side of the patient's chest wall; and
the second end portion includes a bridge member-receiving structure configured to receive the second end portion of any one of the plurality of bridge members, and to allow a distance between the first end portion of the first engaging member and the first end portion of the second engaging member to be adjusted.

17. The strut kit of claim 16, wherein:
at least the first and second opposite end portions of each of the plurality of bridge members comprises a threaded shaft; and
the bridge member-receiving structures of the second end portions of each of the first and second engaging members comprises a threaded bore.

18. The strut kit of claim 16, wherein the first end portions of each of the first and second engaging members includes a concave portion.

19. The strut kit of claim 16, including packaging containing the plurality of bridge members, the first engaging member, and the second engaging member.

20. A method for assembling a strut from a strut kit in accordance with claim 16, comprising:
selecting one of the plurality of bridge members;
attaching the first end portion of the selected bridge member to the bridge member-receiving structure of the first engaging member;
attaching the second end portion of the selected bridge member to the bridge member-receiving structure of the second engaging member; and
adjusting a distance between the first engaging member and the second engaging member by manipulating the location of the bridge member with respect to the bridge member-receiving structure of the second engaging member.
